(19) United States
(12) Plant Patent Application Publication
(10) Pub. No.: US 2017/0231138 P1
(43) Pub. Date: Aug. 10, 2017

(54) Lagerstroemia plant named 'SMLIPINK102'

(50) ;

(71) Applicant: Timothy Wood, Spring Lake, MI (US)

(72) Inventor: Timothy Wood, Spring Lake, MI (US)

(21) Appl. No.: 14/998,758

(22) Filed: Feb. 9, 2016

(51) Int. Cl. A01H 5/02

(52) U.S. Cl. PLT/226

(57) ABSTRACT

A new and distinct Lagerstroemia cultivar named ‘SMLIPINK 102’ is disclosed, characterized by vibrant pink blooms, appearing early in the season. Plants have a compact stature and are very floriferous. The new variety is a Lagerstroemia, normally used for ornamental horticulture purposes.

Latin name of the genus and species: Lagerstroemia indica.

Variety denomination: ‘SMLIPINK102’.

### BACKGROUND OF THE INVENTION

The new Lagerstroemia cultivar is a product of a planned breeding program conducted by the inventor, Timothy D. Wood, in Grand Haven, Mich. The objective of the breeding program was to produce new Lagerstroemia varieties for ornamental commercial applications. The open pollination resulting in this new variety was made during July of 2007.

The seed parent is the, patented commercial variety referred to as Lagerstroemia ‘Red Filli’ U.S. Plant Pat. No. 14,353. The pollen parent is unknown as it was an open pollination breeding program. The new variety was selected July of 2010 by the inventor in a group of seedlings resulting from the 2007 open pollination, in a seedling trial field in Grand Haven, Mich.

Asexual reproduction of the new cultivar was performed by softwood cuttings. This was first performed at Spring Meadow Nursery in Grand Haven, Mich. during July of 2010 and has shown that the unique features of this cultivar are stable and reproduced true to type in 4 successive generations.

### SUMMARY OF THE INVENTION

The cultivar ‘SMLIPINK102’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘SMLIPINK102’ These characteristics in combination distinguish ‘SMLIPINK102’ as a new and distinct Lagerstroemia cultivar:
1. Vibrant pink blooms.
2. Early flowering.
3. Very floriferous.
4 Compact plant stature.

Plants of the new cultivar ‘SMLIPINK102’ are similar to plants of the seed parent, Lagerstroemia ‘Red Filli’ in most horticultural characteristics, however, plants of the new cultivar ‘SMLIPINK102’ differ in the following;
1. Upright/compact habit, whereas the seed parent has an upright and outwardly growing habit
2. Red stem new growth. The comparator has reddish-green new stem growth.

### COMMERCIAL COMPARISON

Plants of the new cultivar ‘SMLIPINK102’ are comparable to the commercial variety Lagerstroemia ‘G2X133271’ PPAF. The two Lagerstroemia varieties are similar in most horticultural characteristics; however, the new variety ‘SMLIPINK102’ differs in the following:
1. New stem growth more red. The comparator has new stem growth is more brown-red.
2. Earlier flowering.

### BRIEF DESCRIPTION OF THE PHOTOGRAPHS

The accompanying photograph in FIG. 1 illustrates in full color a typical plant of ‘SMLIPINK102’ grown in a greenhouse in Grand Haven Mich. Age of the plant photographed is approximately 2 years old from a rooted cutting.

FIG. 2 illustrates a close up of the flowers. The photographs were taken using conventional techniques and although colors may appear different from actual colors due to light reflectance it is as accurate as possible by conventional photographic techniques.

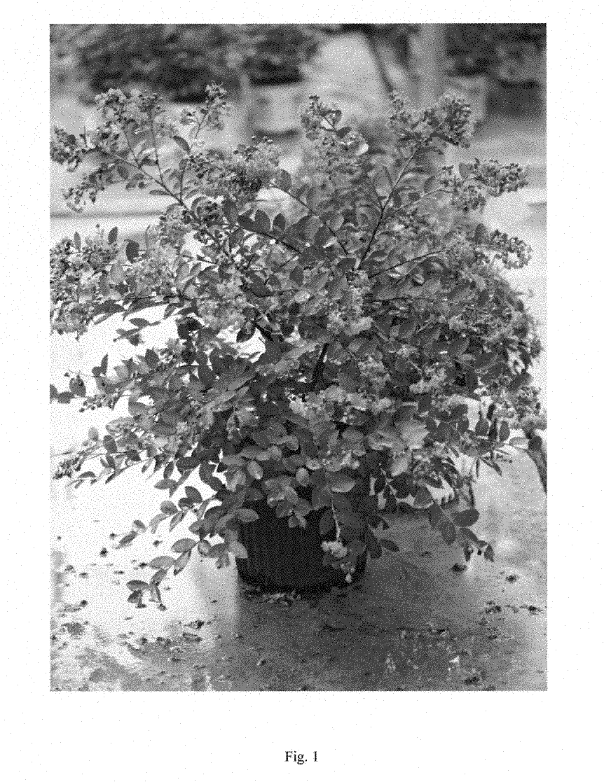

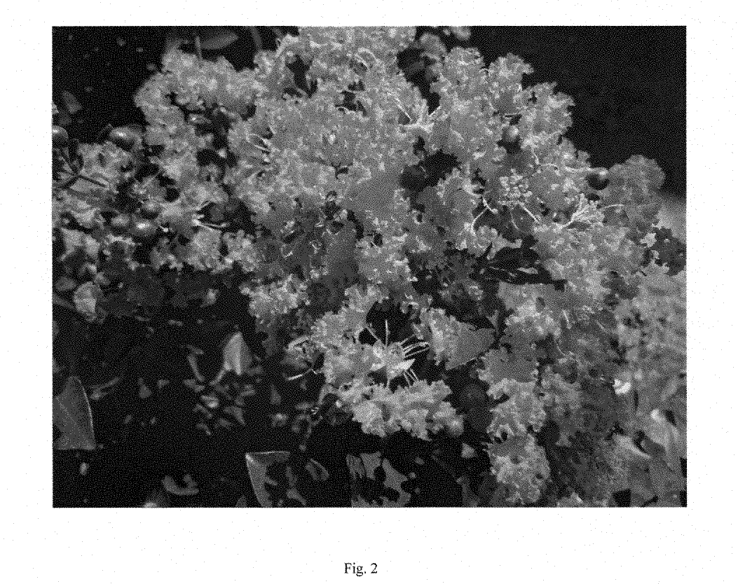

### DETAILED BOTANICAL DESCRIPTION

In the following description, color references are made to The Royal Horticultural Society Colour Chart sixth 2015 except where general terms of ordinary dictionary significance are used. The following observations and measurements describe ‘SMLIPINK102’ plants grown in a poly house during Summer of 2015 in Grand Haven, Mich. The growing temperature ranged from approximately 18° C. to 27° C. during the day and from approximately 5° C. to 10° C. during the night. General light conditions are normal sunlight and numerical values represent averages of typical plant types.
Botanical classification: Lagerstroemia indica ‘SMLIPINK102’
Propagation:
   Type of propagation typically used.—Terminal vegetative cutting.
   Time to initiate roots.—About 14-21 days 24° C.
   Time to produce a rooted cutting.—About 35-42 days at 24° C.
   Root description.—Thin fibrous, not fleshy, freely branching. White in color, not accurately measured with R.H.S. Chart.
Plant:
   Age of plant described.—2-3 years old from a rooted cutting.
   Container size.—3 gallon container.
   Overall plant shape.—Upright.
   Growth habit.—Freely branching outward.
   Plant spread.—85 cm.
   Plant height.—85 cm.
   Growth rate.—Strong.
   Plant vigor.—Good.
   Branching habit.—Basal, some pinching required.
   Length of primary lateral branches.—75 cm.
   Diameter of lateral branches.—1.3 cm.
   Quantity of lateral branches.—7.
   Stem.—Color: Near RHS Brown 200B blending upward to new growth of Red-purple 60A Shape: Square shaped stem Strength: Strong Aspect angle 5-45°Texture: Pubescent, then woody with age. Other: Stem color provides bold contrast to the foliage and flowers.
   Internode length.—1.9 cm.
Foliage:
   Leaf.—Arrangement: Sub-opposite to alternate, single Quantity: Approximately 18 to 28 per main branch. Average Length: New growth: 1.6 cm Old Growth: 5.2 cm Average Width: New growth: 0.75 cm Old Growth: 2.6 cm Shape of blade: Lance-ovate. Apex: Acute Base: Rounded Margin: Entire Texture of top surface: Smooth, waxy Texture of bottom surface: Smooth, waxy Color: Young foliage upper side: Near RHS Greyed-purple 187B blended with Green 137B Young foliage under side: Near RHS Greyed-purple 187B blended with Green 137C Mature foliage upper side: Near RHS Green 137A with Greyed-purple 187B on margin and faded tip. Mature foliage under side: Near RHS Yellow-green 146C blended with Greyed-purple 187B Venation: Type: Pinnate Venation color upper side: Near RHS Greyed-purple 187C Venation color under side: Near RHS Yellow-green 145B Petiole: Average Length: 2 mm Diameter: 1 mm Color: Upper: Near RHS Yellow-green 145B Lower: Near RHS Yellow-green 145B Texture upper and lower: Smooth, coriaceous.
Flower:
   Natural flowering season.—June through October.
   Flower arrangement.—Terminal panicles.
   Inflorescence form.—Panicles.
   Flowers per inflorescence.—25-50.
   Inflorescence size.—Width: 14.5 cm. Height: 12 cm.
   Individual flowers.—Type: Rotate Shape: Whorl of 6 freely attached petals Habit: Outward facing. Diameter: 3.4 cm Length: 4 cm Depth: 1.5 cm Persistence: Self-cleaning Fragrance: Slight to no scent.
   Petals.—Arrangement: Whorled, not fused Number: 6 Shape: Orbicular Margin: Irregular undulate Tip shape: Rounded Base shape: Rounded Length: Average 1.4 cm Width: Average 1 cm Texture: Upper: Soft, smooth, delicate papery Lower: Soft, smooth, delicate papery.
   Color.—When opening: Upper surface: Near RHS Red-Purple 61C Under surface: Near RHS Red-Purple 61C Fully opened: Upper surface: Near RHS Red-Purple 61C Under surface: Near RHS Red-Purple 61C Fading: Upper surface: Near RHS Red-Purple 61C Under surface: Near RHS Red-Purple 61C.
   Bud.—Shape: Obovate Length: 6.5 mm Diameter: 7 mm Color: Near RHS Red-purple 60A blended with Greyed-green 196B.
   Sepals.—Appearance: Healthy, variations of color Arrangement: Unguiculate Shape: Narrowly triangular Tip shape: Acute Base shape: Obtuse Margin: Entire Quantity per flower: 6Length: 5 mm Width: 4 mm Margin: Entire Texture: Upper: Smooth, coriaceous, rugose Lower: Smooth Color: Upper: Near RHS Greyed-purple 184A Lower: Near RHS Red-purple 59A.
   Calyx.—Shape: Unguiculate Length: 5 mm Diameter: 6.5 mm.
   Peduncle.—Length: 1 cm Diameter: 0.5-1 mm Texture. Smooth Color: Near RHS Red-purple 60A Orientation: Upright and outward Strength: Good.
   Pedicel.—Length: 9 mm Diameter: 0.5-1 mm Angle: 5-35°Strength: Good Texture: Smooth Color: Near RHS Red-purple 60A.
Reproductive organs:
   Stamens.—Number: about 32 Filament length: 0.5-1.3 cm Filament color: Near RHS Greyed-red 181C blended with White 155B towards base.
   Anthers.—Shape: Orbital Length: 1 mm Color: Near RHS Yellow-orange 15B Pollen: Color: Near RHS Yellow 12B Quantity: Good.
   Pistil.—Number: 1 Length: 2.2 cm Style: Length: 1.9 cm Color: Near RHS Greyed-yellow 160B fades up to Greyed-red 179C.
   Stigma.—Shape: Globe Color: Near RHS Yellow-green 152B Ovary color: Near RHS Yellow 10C.

### Other characteristics:

Disease/pest resistance.—Neither resistance nor susceptibility to normal diseases of Lagerstroemia.
Garden performance.—Good tolerance of environmental stressors, noted to perform well in harsh winters.

What is claimed is:

1. A new and distinct cultivar of Lagerstroemia plant named ‘SMLIPINK102’ as herein illustrated and described.
